(19) United States
(12) Patent Application Publication
(10) Pub. No.: US 2014/0275687 A1
(43) Pub. Date: Sep. 18, 2014

(54) NON-FISCHER-TROPSCH PROCESS FOR GAS-TO-LIQUID CONVERSION USING MECHANOCHEMISTRY

(71) Applicants: JONES BEENE, MILL VALLEY, CA (US); RONALD CLARK, MORGAN HILL, CA (US)

(72) Inventors: JONES BEENE, MILL VALLEY, CA (US); RONALD CLARK, MORGAN HILL, CA (US)

(21) Appl. No.: 14/202,561

(22) Filed: Mar. 10, 2014

Related U.S. Application Data
(60) Provisional application No. 61/788,877, filed on Mar. 15, 2013.
(60) Provisional application No. 61/817,730, filed on Apr. 30, 2013.

(51) Int. Cl. B01J 19/10; C07C 2/56

(52) U.S. Cl. CPC B01J 19/10; C07C 2/56
USPC 585/717

(57) ABSTRACT

A novel production process is disclosed for the conversion of methane or natural gas, particularly shale gas, into a liquid fuel near the point of origin. The process is notably “non-Fischer-Tropsch” meaning that it does not require oxygen to be admitted into the reactor for supplying thermal energy by partial combustion, which is normally required to split methane. This freedom from high temperature operation and the other demands of an oxygenation process means that higher carbon efficiency is achievable This is made possible with mechanochemistry and “sonic catalysis” that employ kinetic energy to promote the breakdown of methane molecules, the reformation of the resulting carbon-hydrogen fragments, and the rejuvenation of the catalyst surface. A number of liquid fuels can be produced which are easily transported and fully marketable without further processing. Within the range of output products is a liquid solvent which can be used as a substitute “fracking” fluid, which is recoverable as recycled feedstock for further conversion—thus eliminating the problem of water treatment. The reactor can be made more compact, lighter, modular, skid-mounted and fully transportable to the well-head where the gas-to-liquid conversion process reduces the release of natural gas and enables the monetization of stranded or flared gas.

### CROSS-REFERENCE TO RELATED APPLICATIONS

This present application claims the benefit of PPA Serial Number 61′817730, filed Apr. 30, 2013 and PPA Serial Number 61′788877 filed Mar. 15, 2013 by the present inventors.

### BACKGROUND OF THE INVENTION—AND PRIOR ART

This invention relates to the conversion of natural gas, shale gas, and methane to liquid form using a novel non-Fischer Trapsch process involving mechanochemistry and sonic catalysis using easily transportable equipment, and to the monetization of stranded or flared gas by conversion into liquid fuels.

The following is a citation of prior art that presently appears relevant:

### Cited Patent, Publication Date, Applicant, Title

U.S. Pat. No. 3,432,426, Mar. 11, 1969, Laurence D Megel, Oil processor apparatus and method of separating oil mixture components

U.S. Pat. No. 3,478,883, Nov. 18, 1969, Amsalco Inc Acoustic filtration apparatus

U.S. Pat. No. 3,489,679, Jan. 13, 1970 FMC Corp, Ultrasonic clarification of liquids

U.S. Pat. No. 3,490,584, Jan. 20, 1970 Cavitron Corp, Method and apparatus for high frequency screening of materials

U.S. Pat. No. 4,282,100, Aug. 4, 1981, The Sanko Steamship Co., Ltd., Apparatus for reforming fuel oil wherein ultrasonic waves are utilized

U.S. Pat. No. 5,110,443, May 5, 1992, Canadian Occidental Petroleum Ltd., Converting heavy hydrocarbons into lighter hydrocarbons using ultrasonic reactor

U.S. Pat. No. 5,914,027, Jun. 22, 1999, Thermtech A/S, Thermo-mechanical cracking and hydrogenation

U.S. Pat. No. 5,866,751, Feb. 2, 1999, McDermott Technology, Inc. Energy recovery and transport system

U.S. Pat. No. 6,527,960, Mar. 4, 2003, Canadian Environmental Equipment & Engineering Technologies, Inc., Jet pump treatment of heavy oil production sand

U.S. Pat. No. 6,544,411, Apr. 8, 2003, ExxonMobil Research And Engineering Co., Viscosity reduction of oils by sonic treatment

US20100000153, Jan. 7, 2010, Kyrogen USA, Llc., Remote micro-scale GTL products for uses in oil- and gas-field and pipeline applications

WO2003099961A2, Dec. 4, 2003, FMC Technologies, Portable GTL unit and method for capturing natural gas at remote locations

US 2011265737, Nov. 3, 2011, Robetr Ryon, Methods and devices for fuel reformation

WO2004011574A1, Feb. 5, 2004, FMC Technologies, GTL facilities for fixed offshore hydrocarbon production platforms

WO2006012116A2, Feb. 2, 2006, Dijk Technologies LLC Van, Methods for converting natural gas into synthesis gas for further conversion into organic liquids or methanol and/or dimethylethe

WO 2006058107A1 Jun. 1, 2006, Syntroleum Corp, Movable GTL system and process

WO2007127898A2, Nov. 8, 2007, Syntroleum Corp, Method of delivery, replacement, and removal of F-T process

U.S. Pat. No. 8,323,479 B2 Dec. 4, 2012, Saudi Arabian Oil Corp, Converting heavy sour crude oil/emulsion to lighter crude oil using cavitation

U.S. Pat. No. 8,529,858 B2, Sep. 10, 2013, JWBA Inc, Energy efficient, low emissions shale oil recovery process

U.S. Pat. No. 7,078,008 B2, Jul. 18, 2006, ConocoPhillips, Process or converting alkanes to carbon filaments

WO2012118511A1, Sep. 7, 2012, SRI International, Gasification of a carbonaceous material

U.S. Pat. No. 8,293,805 B2, May 29, 2008, Schlumberger Corp, Tracking feedstock production with micro scale GTL units

US201230538 Dec. 6, 2012, Sorokin, Process for the treatment of crude oil and petroleum

US20060180500, Aug. 17, 2006, Sulpha), Inc., Upgrading of petroleum by combined ultrasound and microwave treatments

### BACKGROUND AND PRIOR ART DISCUSSION

Mechanochemistry has been used with coal processing, primarily to crush and to generate heat by grinding, but essentially ignored by the petrochemical industry except as an expedient to upgrade low grade heavy fuel by sonification prior to use in internal combustion. Instead, for over half a century, there have been only incremental improvements and variations of the venerable Fischer-Tropsch (F-T) method. Some of these prior art methods include forms of mechanochemistry, but fall far short of oxygen-free sonic catalysis to break the methane bond.

Sonochemistry and sonication, which are types of mechanochemistry, have been used for converting coal to liquid fuels, and later modified with the addition of natural gas as a part of the feedstock to supply hydrogen. However, oxygen and/or steam have been required in prior art. Essentially these F-T variants have involved adding oxygen in some form at an early stage of processing—and that single requirement is the otherwise insurmountable problem which is solved by a “non-Fischer-Tropsch” method. Oxygen utilization adds complexity and cost, requires large scale for efficient operation and cost reduction, and reduces carbon efficiency. Early oxygenation is ultimately undesirable, and has great comparative disadvantage, even when steam supplies some of the oxygen.

Typical of the patents where mechanochemistry is employed in petroleum for upgrading liquid fuel is U.S. Pat. No. 4,282,100, inventor: Misao Kunishio “Apparatus for reforming fuel oil wherein ultrasonic waves are utilized” which is a basic patent for sonochemistry to upgrade a heavy liquid fuel. This patent was owned by the Sanko Steamship Company and relates increasing the combustibility of fuel oil used in shipping. The patent does not relate to the conversion of methane or other gases to liquid. Typically sonochemistry, as opposed to sonic catalysis, is based on a liquid, solid or colloid which is undergoing cavitation at frequencies in excess of 20 kHz. Sonic catalysis, in contrast, is defined as a high velocity, gas-phase process for gas-phase with entrained solids and liquids), requiring no cavitation—at least not as that term is normally applied. Surface impact on a pitted surface can be called micro-cavitation.

The Kunishio patent was furthered in a claimed improvement: “Methods and devices for fuel reformation” US 20110265737 filed by Robert Ryon. This application discloses methods for reforming fuels by subjecting liquids to ultrasonic energy onboard vehicles powered by combustion engines, to enhance fuel efficiency and/or modify exhaust emissions. It does not apply to gas-to-liquid conversion. Another similar disclosure is U.S. Pat. No. 7,951,288 “Fuel enhancement system for an internal combustion engine” which is method of for treating a hydrocarbon fuel by applying a plurality of shock waves to the fuel at a frequency and intensity such as to increase the combustion efficiency of the fuel. This does not employ methane bond breaking in a jet mill, nor do any prior art mechanochemical devices.

Another prior art process which is related to Fischer-Tropsch, is described in US application 20120046510 “Hydromethanation of a carbonaceous feedstock” in which a steam-integrated and heat-integrated process is used for preparing gaseous products, whereby coal is upgraded into gas and liquids. However, this process requires steam and oxygen and does not employ mechanochemistry. This is representative of several dozen F-T variants, which operate at high temperature, usually 1200° C. and above.

“Gasification of a carbonaceous material” EP 2681292 A1 from SRI International is a related invention which provides a method and apparatus for converting a carbonaceous material to liquid hydrocarbons suitable for use as transportation fuels at lower temperature. No mechanochemistry is used. In the first step the carbonaceous material is converted to a syngas product, which is oxygenated. Methane is preheated to 600° C. and compressed to high pressures using water to replace oxygen, thus reducing the unwanted reaction with the coal, common in the FT spinoffs.

Of interest in prior art is U.S. Pat. No. 8,293,805 “Tracking feedstock production with micro scale gas-to-liquid units,” inventor: Kahn et al. assigned to Schlumberger. This patent teaches a method of tracking production from a natural gas (NG) source that includes the steps of providing one or more micro-scale gas-to-liquid (GTL) units and controlling them. However, the “micro scale GTL” units described are Fischer-Tropsch variants, in contrast to the present disclosure, which is “non-Fischer-Tropsch.” Modular production units, also known as “skid mounted”, are the subject of several expired patents, but none of them involve sonic catalysis or a non-Fischer-Tropsch method. An early patent for transportable GTL production units is McKain, assigned to McDermott Technology: “Energy recovery and transport system” U.S. Pat. No. 5,866,751, now expired. In this disclosure, natural gas is converted at the remote site, using a modified Fischer-Tropsch process to produce non-volatile hydrocarbons. This is an oxygenate system not employing mechanochemistry, nor sonic catalysis, nor jet mills.

The Romanowski and later findings on the spillover capability of nickel and nickel copper-based catalysts is of importance to this disclosure: “Density Functional Calculations of the Hydrogen Adsorption on Transition Metals and Their Alloys” Romanowski at al, Langmuir 1999, Vol 15, 5773-5780. This is an academic paper in which the use of the various alloys has not been reduced to practice.

In this paper, the authors use DFT calculations to predict the dissociation energy of H₂ on Ni-Cu and Ag-Pd alloys. A mixture of 0.625 Ni and 0.375 Cu yields the most energetically favorable conditions for H₂ dissociation. Catalytic power for hydrogen dissociation is in the range of 3 eV. Similar findings from at least three others groups in academia have validated the Romanowski finding but none were reduced to practice in the form of a jet mill reactor, for breaking methane bonds with constant surface renewal of the catalyst.

Over the past thirty years at MIT, the Dr. Sylvia Ceyer and associates have investigated the effects of methane mechanochemistry and the implications of collision induced activation of methane in an ultra-high vacuum, primarily on nickel crystals as the catalyst. This work by the Ceyer group can be seen as an academic validation of the activation mechanics which, in the present invention, has been extended to function in an industrial operational environment and improved with the addition of the Romanowski alloys and sonic catalysis. Over three dozen relevant papers are listed on the Ceyer Research Group website at the MIT Department of Chemistry, which illustrate some of the extreme advantages of this general approach of collision-induced activation—even with a less effective alloy.

Several patents in prior art have recognized the superiority of copper nickel as a spillover catalyst, such as for converting alkanes to carbon filaments. This would include U.S. Pat. No. 7,078,008, assigned to ConocoPhillips. This patent is not related to the present disclosure except for the use of copper nickel catalyst in its role as a dehydrogenator of hydrocarbons.

“Thermo-mechanical cracking and hydrogenation” U.S. Pat. No. 5,914,027 invented by Olav Ellingsen (Original Assignee: Thermtech) is a method for thermo-mechanical cracking and hydrogenation of carbon solids or heavy oils in the presence of water, which is performed in a mechanical fluidized bed. It does permit an overall temperature and pressure which are lower than conventional cracking but does not benefit from sonic catalysis. It also requires water.. The present invention does not employ a fluidized bed, as in Ellingsen, and uses much higher speed media, and does not require water.

For a number of years ultrasound technology has been used to de-sulfurize and hydrogenate crude oil in a process called sonocracking. The sonocracking technology uses ultrasonic energy on a mixture of crude oil and water or crude carbon and water and is an oxygenate process—which distinguishes sonocracking from the present invention. In general, sonocracking is not precisely applicable as prior art, to gaseous mixtures as it is liquid phase. Examples of sonocracking are U.S. Pat. No. 8,323,479 Khan “Converting heavy sour crude oil/emulsion to lighter crude oil using cavitations and filtration based systems.” This is a low-temperature process using cavitation of a water and carbon. U.S. Pat. No. 8,336,621 invented by Bunger: “Energy efficient, low emissions shale oil recovery process” provides a non-oxidative pyrolysis process for the recovery of liquid from oil shale or tar sands but does not employ mechanochemistry.

US 20060180500 Gunnerman “Upgrading of petroleum by combined ultrasound and microwave treatments” refers to petroleum liquids and does not involve methane bond breaking or a jet mill. A similar disclosure is US 20120305383 Sorokin: “Process for the treatment of crude oil and petroleum products” which provides a process for the treatment of crude oil using ultrasound vibrations and an electromagnetic field but involves crude oil without methane and is an oxidative process.

In general, the older F-T gasification processes require elemental oxygen to maintain a temperature of ˜1,400° C. which has been gradually reduced over the years. However, even most recent processes operate with a temperature of ˜600° C. and require the presence of steam and high pressures that demand an expensive thick-walled stainless steel reactor costing far more in overhead.

An oxygen-free process is necessary for a substantial improvement in terms of “carbon efficiency” which is the percentage of carbon in the output stream, compared to the carbon available in the input. The use of mechanochemistry in this embodiment eliminates the need for oxygen and can operate at 400° C. and at low pressure in an oxygen-free process. Low alkane mixed-liquid products—which require no oxygen at all, are the primary products of one embodiment. If an oxygenated alcohol fuel is desired, in another embodiment, addition of the hydroxyl is delayed until after the first stage (that produces C4 butane/butane), producing an alcohol fuel such as butanol as the end product in the second stage. The final conversion of mixed C4 gases (butane/butene and isomers) to alcohol is optimized by sonochemistry—thus lowering the net energy required to a minimum and increasing the carbon efficiency of the feedstock to the maximum.

### BRIEF SUMMARY OF THE INVENTION

A mechanochernical process to convert natural gas, shale gas or methane into liquid hydrocarbon products is accomplished with kinetic energy produced by one or more jet mill reactors with rotating blades. Feedstock, consisting of gas molecules and carbon particles, is injected into the jet mills and accelerated to create high velocity impact on a catalyst surface, causing breakdown or dehydrogenation of methane while providing continual resurfacing of the catalyst surface, thus eliminating the need for oxygen and lowering the thermal requirement for splitting methane while keeping the catalyst activated. The use of mechanochemistry in this process enables production of a small, portable reactor that can be located and operated near the gas source and can be operated with multiple reactors for larger scale operation.

### ADVANTAGES OF THE INVENTION

Accordingly, several advantages are as follows: to provide efficient conversion of natural gas at the gas source, to eliminate costly process in existing petrochemical reactor systems, to provide low cost operation of a gas conversion system, to reduce flaring of natural gas at the gas fields, to produce liquid fuels that are easily transported, to be scalable from small, portable units to larger sizes and multiple units, and to produce lower cost fuels.

### BRIEF DESCRIPTION OF THE DRAWINGS

FIG. 1 shows a cross section of a jet mill configured as in one embodiment with feedstock entering at the top and the converted products exiting at the bottom.

FIG. 1A shows one embodiment of a propeller that accelerates the feedstock.

FIG. 2 shows one embodiment of the gas-to-liquid process.

FIG. 3 shows an embodiment of second stage that can be operated with the process shown in FIG. 2 to produce an oxygenated fuel.

FIG. 4 shows an alternative embodiment of a gas-to-liquid process.

### DETAILED DESCRIPTION OF THE INVENTION

In one embodiment, shale gas with substantial natural liquid content is converted into a marketable liquid by the synergy of mechanochemical and catalytic methods, which are the disclosed novelty of the process. The mixed-liquid output fuel product can be sold locally as fuel oil, furnace fuel, or boiler oil replacements, or as light alkanes for blending with gasoline. These products are marketable directly to local customers in every state without further processing. In a second embodiment of the invention, which is a Methane-to-Butanol process—water is added in a second stage to convert butane and C₄ isomers into butanol, which is a desirable liquid transportation fuel; superior to gasoline in many respects, or blended as a “gasohol.” In a third embodiment, two or more reactors are employed, with added carbon and different catalysts, to produce various higher carbons such as hexane and octane.

The rapid expansion of the shale gas industry in recent years, the growing glut of natural gas supplies, and the problem of stranded and flared gas, have combined to raise the interest in liquid fuels made from methane. But that interest is tempered by the extreme capital cost and regulatory obstacles of fixed petrochemical facilities which require a version of the Fischer-Tropsch process—plus the further cost and delay of dedicated gas pipelines for transport. An oxygen-free process, which can be called non-Fischer-Tropsch, is highly preferred due to lower operating temperature, lower loss of feedstock, simplicity, greater control and lower overhead. However, no process in prior art has been robust enough to completely dispense with oxygen at an early stage, due to lack of an effective catalyst along with the issue of catalyst passivation dynamics, among other problems.

Mechanochemistry is most often seen in industry in ball-milling or sonication (ultrasound) applications. Even though the basic process is well-known, mechanochemistry has seldom been employed in basic petrochemical processing. The key discovery which has enabled this breakthrough is the combination of mechanochemistry with a superior bulk catalyst. The catalyst in this invention is one which works well when its surface layer is continually reactivated by kinetic activity and abrasion (shear dynamics). Therefore, this catalyst is employed efficiently in typical prior art petrochemical processing—which generally involve catalyst beds with the catalyst supported on a porous ceramic bed. The synergy of the mechanochemistry system derives from the fact that the same shear forces which keep the catalyst active also supply some of the energy necessary to split the strong methane bond (435 kJ/mol) on the catalyst surface and provide an environment for the formation of higher alkanes. It has been shown that that methane can be dehydrogenated with as little as 60 kJ/mol using mechanochemical techniques in laboratory tests operating in an ultra-high vacuum. In addition, a major advantage of having an unsupported, electrically insulated and electrically conductive catalyst located inside the reactor—is the ability to provide electrical charge directly to the catalyst, which is essentially impossible in traditional chemical reactors.

Therefore, the advance which is allowing higher efficiency than Fischer-Tropsch is found in mechanochemistry. In one embodiment, this is accomplished through the alteration of the known jet mill of prior art into a pressurized catalytic chemical reactor. One key modification is the addition of a replaceable reactor liner, composed of a relatively low cost, structurally strong nickel alloy, which is itself a vastly superior spillover catalyst. Other modifications include the addition of temperature and pressure control, and the capability to establish voltage gradients in the reactor. The so-called Romanowski spillover catalyst group of copper-nickel alloys is important to this invention because, when combined with sonic velocity shear forces, these alloys make it feasible to break the strong methane bond at comparatively low processing temperature. At the same time, the normal kinetics of jet mill operation operate to keep the catalyst surface active—but at the expense of surface wear, forcing the reactor liner to become “sacrificial” over time. Periodic replacement of the catalytic liner, with minimal down time, is designed into the system, as part of the novelty of the process.

In another embodiment, a different catalyst can be used for a second adjoining mill, where the catalyst is better suited to aid in polymerization of carbon species into longer chains, in the case of favoring a particular end products such as octanes—but at least one reactor-mill in this embodiment will contain a nickel copper alloy. Because two or more shuttling reactors can be employed in this process—two different catalysts can be used under differing parameters for better control and selectivity of the output products, enabling the production of target liquid products including transportation fuels. These blended fuels do not require further treatment or distillation.

The jet mill impellers provide acceleration of a mixed feedstock to sonic velocities, typically in excess of 300 meters per second. Thus we use the name: “sonic catalysis” to describe a mechanochemical process capable of breaking the methane bond efficiently and providing the ability to continually rejuvenate the catalyst surface. This is very different from “sonication”.

The volatility gradient of the various in-process gases makes rapid separation and enrichment possible, using only a simple cyclone or vortex separator, allowing an optimization of the system using computer control and solenoid operated valves. This ease of separation is due to dramatic change in volatility and density due to phase-change from gas to liquid of pressurized C₃ propane/propene molecules and C₄ butane-based molecules which are gaseous without pressure, but are liquids when under modest pressure. This is a most fortuitous physical property of the in-process feedstock, which has been fully optimized in this novel system.

Oxygenated end-products as well as non-oxygenated or mixed fuels, all within the C₄-C₈ range of carbon can be produced by slight process extensions to this process. A single stage process for non-oxygenated output can be complemented with a final oxygenation stage, which also uses sonic catalysis but no elemental oxygen is required-only water. The early embodiments of the present invention are focused on a mixed light alkane liquid which will serve as a replacement for fuel oil, but which can be burned in a converted diesel engine, of an optimum multi-fuel design with added lubricant. It is realized that the early markets for the light alkanes could be economically saturated geographically, and consequently certain segments of the larger transportation fuel market are envisioned as being accessible with the higher value liquids produced by this system, such as butanol.

However, the lighter alcohols: methanol and ethanol are measurably inferior as ecologically sound transportation fuels, but butanol, either alone or blended is a desirable end-product for commercialization of shale gas, stranded natural gas, land-fill gas or methane clathrate in many locations. Other marketable or usable chemical products, including heavier hydrocarbons from added coke or coal, can be produced in the same system with additional reactors. Also, this process can produce substitute solvents and hydrocarbon liquids for use in the so-called tracking process for oil and gas extraction. These substitute tracking fluid products are recoverable and reusable. Direct gasoline substitutes like mixed C₆ to C₈ can be produced without an oxygenation step with an enhanced control system in a multiple jet mill configuration. This C₆ to C₈ process produces a limited range output and offers a limited flexibility in the purity of the end-product, and has dependence on the particular characteristics of each well site.

It has been appreciated for some time in prior art that butanol is superior to gasoline in the typical internal combustion engine in providing higher efficiency due to more complete combustion with less pollution. In addition, butanol from shale gas is less volatile, safer to handle, less corrosive and can be blended with butanol produced biologically. The fermented version: biobutanol—has been the major focus of research for providing an alternative to ethanol. Butanol has not previously been made efficiently from methane, shale gas, or a combined coal process for a variety of technical reasons that involve high heat and energy and cost requirements.

Even with added carbon, traditional methane conversion processes are inefficient, in terms of carbon efficiency, when oxygen must be added initially. These prior art processes involve carbon monoxide chemistry which creates pollution risks. Many mature production processes in the Fischer Tropsch category are overly complicated, despite the numerous advantages of stream-lining for a simplified commercial fuel product range. Existing systems have high energy cost for some outputs, primarily because catalysts and conversion systems have not been optimized to produce four-carbon (C₄) or higher molecules using the added control inherent in mechanochemistry. Sonic catalysis, as defined herein, can be optimized and controlled to produce a high proportion of C₄ molecules preferentially and this is impossible with a thermal oxygenate processes.

The input feedstock for production of butanol, as an example, is derived from approximately 45-55% shale gas by weight, 30-40% crude carbon and 15-20% water. These proportions are variable by design, with products such as octane requiring no water and higher levels of crude carbon. The raw material equivalent cost can be as low as 30 percent of the wholesale value of the output, based on actual 2013 prices, since little feedstock is lost to oxidation. The most costly operational item is fuel for the gen-set but that is not an out-of-pocket expense, since the ICE will be adapted to run on one of more of the feedstock inputs.

The methane market could be in a relative glut supply position with low cost or an extended timeframe, compared to transport fuel. Therefore, a shale gas well operator should be able to double his net return in as little as two years by converting methane to butanol and selling it locally to gasoline blenders, instead of processed methane to traditional markets where it must be transported as a gas. This ability to monetize instead of flare has significant ecological advantages. A marginal or stranded shale gas production site becomes amenable to commercial exploitation, even if gas produced by the well will play-out in a matter of months. The system eliminates the delay of constructing a permanent factory, pipeline or other extensive infrastructure. Each skid or mini-plant of this invention is estimated to produce approximately 2-2.5 million gallons of liquid fuel per year, in a preferred embodiment. That embodiment will typically be designed around the largest prime mover ICE (internal combustion engine) which is available at low capital cost.

Electrical usage is substantial and benefits greatly from on-site production without the complication of a grid connection. Electrical power and cogeneration of heat is superior to internally generated heat input, for better system control and lower pollution. A pneumatic type of transport is provided for the local movement of carbon to each skid from a storage silo. The carrier gas for this transport system is the dry shale gas used in the process. No air or oxygen is permitted in the production system,

### OPERATION OF THE INVENTION

The following narrative will contain preferred embodiments of the invention but is not intended to limit the scope of the disclosure. Details of the Jet Mill reactor, with replaceable liner, are shown in FIG. 1. A conceptual drawing of the GTL system with major components is shown in FIG. 2. FIG. 3 shows an optional second stage, in which added water and sonication produces an oxygenated fuel-butanol. FIG. 4 shows an alternative embodiment of a gas-to-liquid process

In the modular embodiment of the invention which is optimized for setup and implementation at remote shale gas production sites, the GTL (gas-to-liquids) processing hardware, as described below, will be mounted on one or more skids which can be ruggedly designed for truck transport on secondary or unpaved roads. The skids will be capable of long-term outdoor operation when lifted from the truck and mounted on a compacted foundation, poured concrete or piers—without the necessity of an enclosed factory setting. Alternatively a complete truck chassis can be modified as a mini-GTL plant.

The main components of the skid will include at least one jet mill reactor (FIG. 1), which can be powered by an electric motor (101), or alternatively which can be directly driven from an adjacent internal combustion engine using a PTO mechanism (power take-off). Each mill, and there can be many mills per drive motor, will consist of a reactor vessel (102) with a replaceable liner (103) and with an internal impeller (104) with a pressure tolerant bearing (105) and an intake port (106) and exhaust port (107) both of which ports are operated by solenoid control (108). Each impeller will consist of blades or discs (104) which spin at high speed inside the reactor body. The impellers operate to accelerate the shale gas media (mixed media containing gas, solids and liquids) in the radial vector from the vertical axis of rotation. The velocity of the media is typically 330 meters per second or higher, but may operate at lower speeds.

The intake port will generally be aligned with the axis of the reactor and the exhaust port will be located near the bottom of the reactor which is an inverted cone. The acceleration of feedstock is centrifugal. When both ports are closed, the mixed feedstock media recirculates through the discs, impacts the catalytic wall, is expelled generally downward and upward, after it is sucked through openings in the discs located near its axis—in a repeating cycle of many passes per second. Ideally, with a mean path of slightly over one meter, the feedstock media can be recirculated up to one thousand times in a 3-4 second cycle. This allows a high net rate of conversion to liquid, even when the rate of initial bond breaking is relatively low per pass.

At a predetermined period of operation, based on a library of control parameters, valves will open and the jet mill reactor will dump the process mix into a separation device. The separation device will operated by volatility and phase change—so as to remove liquid phases as soon as a substantial quantity is made, and thus to prevent polymerization. The working feedstock of higher volatility is then returned to the jet mill, usually mixed with an addition of unprocessed feedstock. The process can be called batch-continuous with individual completed cycles overlapping—typically in time range of seconds or tens of seconds.

The impact zone or target for the high speed gas is a replaceable catalytic liner (104) which serves as the main catalyst for splitting the methane molecule and the gas conversion process. This liner is subject to constant wear from kinetic impact and is replaceable with a new liner on scheduled maintenance. All the metal in the old liner is salvageable.

The feedstock will typically be shale gas with high liquid hydrocarbon content, added carbon in powder form and, in some embodiments, a percentage of argon, or any other heavier inert gas which is retained in the system. The function of the argon is to provide added mechanochemical kinetic impact without a chemical reaction. Argon alone will provide adequate shear for some degree of methane dissociation.

In FIG. 2, a process for one embodiment is shown. A dedicated genset (201) is be provided for electrical power to the jet mill (202). The genset can be mounted on the same skid as the jet mill or in on an adjacent skid. The genset will operate in co-generation mode to supply both electrical power and process heat to the jet mill reactor. Ideally the genset will be based on a mass produced, multi-fuel, Internal Combustion Engine (ICE) which can be modified to burn either raw shale gas in some circumstances, or the liquid output of the system in other circumstances, or shale gas liquids in other circumstance, or methane alone—and will be capable of burning excess hydrogen gas which is separated from methane as a part of the GTL process.

In normal operation, shale gas (203) along with entrained gas liquids is first subjected to computerized gas analysis (204) to continuously monitor the composition of the input gas mix. The gas is then cleaned of contaminants, including sulfur and water by a scrubber device (205). This is done in the usual fashion known to the industry. The prepared gas can be temporarily stored under pressure near the well-head, prior to being ported into the GTL jet mill processors. The gas supply also serves as the carrier gas for pneumatic feeding of carbon powder, when carbon is added. The gas content analysis is fed to a control system (206) which is remotely accessible by LAN or smart phone. The computerized control system has a database of parameters, based on known processing variable for various input mixtures which have been encountered before and with reference to the desired output; but with the flexibility to create new parameters. The control system operates by triggering solenoid operated valves (207) which move the batch through the system based on the results of ongoing analysis and a stored library of control parameters for each selected output liquid.

The primary mode of control of finished processing involves the use of phase change from C₁ to C₄ and higher hydrocarbons, and the physical property of densification in going to the liquid phase. The volatility gap in gas phase between methane and higher carbon gases allows for easy separation by vortex centrifugation. One or more separation devices (208) work via a timed sequence, and by phase-change and density gradient so as to separate liquid output from gases to be recycled during the jet mill sequential batch operation. The separation devices can be of the cyclone-type or vortex-type and are solenoid controlled. Residual solid (209) are collected for later processing.

Following any initial batch of feedstock, there will seldom be a complete conversion during the first cycle but the residual feedback from one cycle is supplemented by new feedstock on the next cycle, which can vary in length from several seconds to minutes. Since the residual process gas contains radicals, ions and metastable intermediates, this composition reduces the required energy to split more methane. One or more in-process storage tanks (210) can be provided for each jet mill. Electrical charge can optionally be added to the catalyst in the jet mill as a further control resource.

This invention is an automated process which can run full time—24/7 with minimal human supervision, depending heavily on computer control, monitoring with feedback, and a library of parameter routines. Ongoing gas analysis (204) is provided by IR, laser or other well-known mass spectroscopy methods.

This GTL system can be operated with well-head shale gas alone, when sufficient liquids are naturally entrained in the gas, but operated with added solid carbon as well. Added carbon in the form of crushed coke, coal, charcoal, or any other powder form is stored in a hopper (209) and transported to the jet mill intake using process gas as the pneumatic transport mechanism. In the event of inability to obtain powdered carbon of known properties, shale oil from adjacent wells or pyrolyzed shale oil can be substituted.

The final liquid output is analyzed (211) and stored for truck or rail transport out of the production zone. The output can go directly to local markets or be sold for blending as a mixed liquid. Further processing for preselected liquid transportation products can be done at a nearby refinery if one is available, but is generally not required. Four-carbon olefins or alkenes, especially butene, butane, and their isomers and intermediates—can be favored by the production and control parameters. These are often in demand for further processing, and are selectively removed by volatility gradient when their concentration reaches a preprogrammed level.

A non-oxygenated fuel with more than four carbons, such as light alkanes or hexane, heptane and octane mixes, are easily converted from C₄ into satisfactory liquid fuels which can be available for distribution to customers without further processing. The volatility separation devices (206) are standard processing equipment. At the operating pressure and temperature, the desired output products are in liquid phase and all other species are either gas or solid to be retained in the system. Thus, there is inherent ease of separation which is not generally employed in prior art. Solids, including minerals, sand and clay can be purged periodically.

A second stage process, shown in FIG. 3, is part of the present disclosure, which in sonochemical, employing ultrasound—and can be employed to produce alcohols and oxygenated end products. A sonicator or ultrasound condenser (301) can consist of a rotary pump with cavitation dimpling on the impeller in the simplest incarnation. A “hydrosonic TM” or shock-wave pump is an example, but traditional ultrasound horns can also be employed. The sonification happens in a pressurized liquid phase, combining C₄ and higher feedstock from the first stage, described above, with water (302), pressure, sonication (ultrasound sonification) and optional RF input. A vortex separator (305) removes the liquids and returns any remaining gas phase to the ultrasound condenser, No added heat or elemental oxygen is required to produce a mixed alcohol blend in the form of the heavy alcohols, preferably butanol (306). Thus, the products are market-ready in two steps.

A second embodiment is shown in FIG. 4 in which feedstock is compressed outside the jet mill reactor. In this lower cost, lower output configuration, pressurized feedstock is injected into a reactor (401) through high speed nozzles (402) at sonic velocities striking a solid catalyst (403) which may be stationary or rotating. Feedstock which can consist of methane, shale gas with entrained liquids, or gas molecules with added carbon particles, as in the jet mill of the first embodiment, is accelerated to sonic velocities.

The high velocity impact of the feedstock on the catalyst surface enables the breakdown of the methane molecules and causes continual resurfacing of the catalyst surface. As the carbon molecules breakdown and reform into higher carbon alkanes and alkenes, heaver liquids are removed by volatility gradient or phase change, using one or more processes which can include: cooling, repressurization, and vortex separation.

Feedstock (404) entry and gas and liquid extraction are computer controlled (405) via solenoid-operated valves (406). Reactor contents are temperature monitored with thermocouples (407) and controlled with a heater (408) and a cold trap (409) or cooler. A compressor (410), along with control valves, controls the gas pressure (411) and velocity. Gas conversion is monitored with a gas analyzer (412) and by analysis of the collected liquid products (413). The solid catalyst can be electrically charged.

When butanol is the desired end product, the two steps of the overall process can be timed in unison so that there is little delay or need for storage between steps. Avoiding high heat and increasing control options allows the operator to limit polymerization and decrease energy usage. Unlike all prior reforming or cracking processes, no elemental oxygen is required anywhere in the system as water supplies the hydroxyl during the second step. Butanol will derive solely from water or steam addition, catalysis and sonochemistry. The input energy including the frequency, intensity, and timing of RF radiation and ultrasound can also be controlled by computer with template modeling for maximum yield of the desired liquid fuel.

In this fashion, a semi-permanent but fully contained production facility can be placed in operation at, or near an operating shale gas platform within days of initial wellhead operation and regulatory approval, thus avoiding the necessity for a gas pipeline or large gas storage facilities. Also avoided are oxygen sources, a large water supply, or new electrical lines.

The multi-fuel adapted genset (201) will be a major cost component of the process. Thus, it will be adapted from a mass-produced engine, which is typically a diesel with output in the range of 200 kilowatts and up (electrical) or 400 kilowatts (thermal and electrical) as a cogenerator (212) of heat and electricity. Multiples of the skid mounted mini-plants are feasible at any location without reliance on a grid supplier of electricity or a pipeline. The modular units are moveable and reusable sequentially—when shale gas is exhausted at any site.

The higher end product from this process is fuel grade butanol, which is intended to be sold to local fuel blenders and distributors, to be blended with gasoline for an improved “gasohol” or to be used alone when regulations permit. Biobutanol is approved by the EPA to be blended at the 11% level (16% is on the horizon). In the case of octane and cyclic carbon liquid fuel blends, no permit should be required as these hydrocarbons are already found in commercial gasoline.

The jet mills (101) are modified by the incorporation of the Monel spillover catalyst as an alloy—directly into replaceable liner walls (102) and/or in the impeller of the mills (105) or in the outer wall of the jet mill (402). The impellers of each jet mills can be constructed of discs or blades or bladed discs of a catalytic alloy. The Monel alloy liner wall, effectively removes protons from methane at approximately twice the efficiency (in catalytic power) as other spillover catalysts, such as pure nickel, palladium or palladium silver. The mills are modified to operate at internal pressurization, which is necessary for a number of reasons including separation or gases by volatility gradient and phase change. The pressure can be moderate—less than 10 bar.

Initially, methane and solid carbon particles are rapidly recirculated, resulting in the serial addition of hydrogen to carbon molecules—first going to acetylene/ethylene/ethane then to propene/propane and then to butene/butane as well as various stable and metastable isomers. The control parameters are monitored to determine when a favorable equilibrium proportion of butene is maximized in this process gas. Parameter templates will have been determined in advance. Valves are then remotely switched to port the synthesis gas to a volatility gradient separator. Hydrogen sulfide and solids removal can be accommodated in the same fashion at an earlier stage. Volatile fractions are returned to stage-one processing and solids are collected as process ash.

In the second stage which is provided for the production of butanol, but is not otherwise necessary, sonochemistry converts C₄ (as butene/butane) to butanol in the approximate same cycle period which is utilized in stage-one. This stage is also pressurized, so that technically the reactor contents are liquid equivalent from the start. The main function of stage two is to sonicate water so that hydroxyl ions are added, via catalysis, to produce alcohol.

A rotary cavitation pumping device, also known as a hydrosonic pump or shockwave pump can be used to assist in this transformation to butanol. RF (radio frequency) input can also be provided at a resonant frequency or complex waveform to further enhance the desired product. At the termination of the second stage, the lighter volatiles including hydrogen are separated and returned to either the first or second stage, dependent on volatility. The presence of smaller amounts of other liquid oxygenated hydrocarbons such as C₅ amyl alcohols is deemed to be acceptable since the product is a combustion fuel. The butanol fuel is of relatively low volatility and is easily moved to storage or transport to a gasoline fuel wholesaler or blender.

1. A mechanochemical process to convert natural gas, shale gas or methane to liquid hydrocarbon products—which process is accomplished with kinetic energy produced by one or more jet mill reactors which are fitted with rotating impellers, and, in which the reactor contains a catalyst that is provided in the form of the structural wall or as a replaceable liner of the reactor; and in which shear forces of accelerated feedstock create high velocity impact on the catalyst surface, causing breakdown of methane and continual resurfacing of the catalyst surface, thus lowering the thermal requirement for splitting methane while keeping the catalyst activated.

2. The process of claim 1 where a crude source of carbon, in the form of a powder, particulate, vapor, colloid or liquid is added to the natural gas feedstock for the reactor; which carbon source can be derived from coal, coke, charcoal, shale oil, crude oil, pyrolyzed oil, refinery sludge, or equivalent crude carbon.

3. The process of claim 1 wherein the high velocity impact of gas molecules and/or carbon particles creates transient high temperature, high pressure microvolumes on or near the surface of the catalyst in such a manner that the microvolumes promote the dehydration of the methane molecule and formation of carbon-carbon bonds by partial adsorption leading to higher alkanes and intermediate hydrocarbon molecules.

4. The process of claim 1 wherein electrical charge, especially a low voltage, high current negative charge is applied directly to an electrically isolated catalyst in order to promote an increased rate of spillover dehydrogenation and methane bond-breaking.

5. The process of claim 1 where a predominantly four carbon (C₄) output product, which is a mixed butane/butene gas with entrained liquids, is converted into a mix of heavy alcohols, primarily butanol, using water sonochemistry as an additional step.

6. The process of claim 1 where the gas-to-liquid conversion components are provided in a transportable format, such as mounted as a complete working module on a transportable skid, rail car or truck bed, so as to be implemented near the shale gas production well as a modular mini-factory, along with cogeneration of thermal and electric power as part of the process.

7. The process of claim 1 where a volatile liquid solvent or a heavy gas entrained with liquid hydrocarbons is produced as a substitute for water-based liquids—to be further employed specifically as a reusable “fracking” fluid for the purpose of facilitating shale oil and gas extraction; which fluid is recoverable along with resultant shale gas and can be processed again within the system.

8. The process of claim 1 wherein a predominantly nickel-copper alloy, such as the alloy known as Monel, is used as the combined catalyst and structural liner or wall of the jet mill reactor; and additionally where such alloy is fabricated as the impeller disks of the jet mill, which is generally of a multi-disk design of prior art but in which the impeller disks are fabricated of catalyst.

9. The process of claim 1 where a second jet mill is used to process the output of the first jet mill and uses a catalyst with different material composition and conversion properties to promote the formation of selected output products.

10. The mechanochemical process of converting methane to liquid wherein a gaseous feedstock, which can contain added carbon, is pressurized externally to the reactor and injected into a reactor via one or more entry nozzles, striking a stationary or rotating catalyst surface at near normal right angles, causing breakdown of methane and continual resurfacing of the catalyst surface, thus lowering the thermal requirement for splitting methane while keeping the catalyst activated.

11. The process of claim 10 wherein a predominantly nickel-copper alloy, such as the alloy known as Monet, is used as the catalyst in the reactor and in which the catalyst can be electrically isolated and charged electrically in order to promote the breakdown of the methane molecule.
